(19) United States
(12) Patent Application Publication
(10) Pub. No.: US 2024/0065881 A1
(43) Pub. Date: Feb. 29, 2024

(54) FLUID COLLECTION ASSEMBLIES INCLUDING ONE OR MORE LEAK PREVENTION FEATURES

(71) Applicant: PUREWICK CORPORATION, Covington, GA (US)

(72) Inventors: Melody Mei Hee Kuroda, Durham, NC (US); Hannah Rose Kriscovich, Marietta, GA (US); Brian Murphy, Baltimore, MD (US); Michael Bielawa, Baltimore, GA (US); Florence Stevenson, Baltimore, MD (US); Carolyn Rose, El Cajon, CA (US); Charlotte Lux, El Cajon, CA (US); Alexis Dorsey, El Cajon, CA (US); Alisa Ratanen, El Cajon, CA (US)

(21) Appl. No.: 18/249,577

(22) PCT Filed: Oct. 19, 2021
(86) PCT No.: PCT/US2021/055515

§ 371 Date: Apr. 19, 2023

Related U.S. Application Data
(60) Provisional application No. 63/094,594, filed on Oct. 21, 2020.

(51) Int. Cl. A61F 5/455; A61F 5/44

(52) U.S. Cl. CPC A61F 5/455; A61F 5/4401; A61F 5/4404

(57) ABSTRACT

Embodiments disclosed herein are directed to fluid collection assemblies including one or more leak prevention features, systems including the same, and methods of using the same are disclosed herein. An example fluid collection assembly includes a fluid impermeable barrier at least defining a chamber, at least one opening, and a fluid outlet. The fluid collection assembly also include at least one porous material disposed in the chamber and one or more leak prevention features. The leak prevention features may include at least one of a shape of the fluid collection assembly that improves fit between the fluid collection assembly and a patient, at least one absorbent material at least partially disposed outside of the chamber, a base configured to have a patient disposed thereon, or one or more features that allows a length of the fluid collection assembly to change.

### BACKGROUND

A person or animal may have limited or impaired mobility such that typical urination processes are challenging or impossible. For example, a person may experience or have a disability that impairs mobility. A person may have restricted travel conditions such as those experienced by pilots, drivers, and workers in hazardous areas. Additionally, sometimes urine collection is needed for monitoring purposes or clinical testing.

Urinary catheters, such as a Foley catheter, can be used to address some of these circumstances, such as incontinence. Unfortunately, urinary catheters can be uncomfortable, painful, and can lead to complications, such as infections. Additionally, bed pans, which are receptacles used for the toileting of bedridden patients are sometimes used. However, bedpans can be prone to discomfort, spills, and other hygiene issues.

### SUMMARY

Embodiments are directed to fluid collection assemblies including one or more leak prevention features, systems including the same, and methods of using the same are disclosed herein. In an embodiment, a fluid collection assembly is disclosed. The fluid collection assembly includes a fluid impermeable barrier that at least defines a chamber, at least one opening configured to be positioned adjacent to a urethral opening, and a fluid outlet. The fluid collection assembly also includes at least one porous material disposed in the chamber and extending across the at least one opening. The fluid collection assembly further includes one or more leak prevention features.

In an embodiment, a system is disclosed. The system includes a fluid collection assembly. An example fluid collection assembly includes a fluid impermeable barrier that at least defines a chamber, at least one opening configured to be positioned adjacent to a urethral opening, and a fluid outlet. The fluid collection assembly also includes at least one porous material disposed in the chamber and extending across the at least one opening. The fluid collection assembly further includes one or more leak prevention features. The system also includes a fluid storage container and a vacuum source configured to supply a suction force to the fluid collection assembly. The chamber, the fluid storage container, and the vacuum source are in fluid communication with each other via at least one conduit.

Features from any of the disclosed embodiments may be used in combination with one another, without limitation. In addition, other features and advantages of the present disclosure will become apparent to those of ordinary skill in the art through consideration of the following detailed description and the accompanying drawings.

### BRIEF DESCRIPTION OF THE DRAWINGS

The drawings illustrate several embodiments of the present disclosure, wherein identical reference numerals refer to identical or similar elements or features in different views or embodiments shown in the drawings.

FIG. 1A is an isometric view of a fluid collection assembly, according to an embodiment.

FIG. 1B is a cross-sectional schematic of the fluid collection assembly taken along plane 1B-1B as shown in FIG. 1A.

FIG. 2A is an isometric view of a fluid collection assembly, according to an embodiment.

FIG. 2B is a cross-sectional schematic of the fluid collection assembly taken along plane 2B-2B as shown in FIG. 2A.

FIG. 3A is an isometric view of a fluid collection assembly, according to an embodiment.

FIG. 3B is a cross-sectional schematic of the fluid collection assembly taken along plane 3B-3B as shown in FIG. 3A.

FIG. 4A is an isometric view of a fluid collection assembly, according to an embodiment.

FIG. 4B is a cross-sectional schematic of the fluid collection assembly taken along plane 4B-4B as shown in FIG. 4A.

FIG. 5A is an isometric view of a fluid collection assembly, according to an embodiment.

FIG. 5B is a cross-sectional schematic of the fluid collection assembly taken along plane 5B-5B as shown in FIG. 5A.

FIG. 6A is an isometric view of a fluid collection assembly, according to an embodiment.

FIG. 6B is a cross-sectional schematic of the fluid collection assembly taken along plane 6B-6B as shown in FIG. 6A.

FIG. 7A is an isometric view of a fluid collection assembly, according to an embodiment.

FIG. 7B is a cross-sectional schematic of the fluid collection assembly taken along plane 7B-7B as shown in FIG. 7A.

FIG. 8 is an isometric view of a fluid collection assembly, according to an embodiment.

FIG. 9A is an isometric view of a fluid collection assembly, according to an embodiment.

FIG. 9B is a cross-sectional schematic of the fluid collection assembly shown in FIG. 9A taken along plane 9B-9B.

FIG. 10 is an isometric view of a fluid collection assembly, according to an embodiment.

FIGS. 11A and 11B are isometric views of a fluid collection assembly exhibiting a first length L₁ and a second length L₂, respectively, according to an embodiment.

FIG. 12 is an isometric view of a fluid collection assembly that is configured to change a length thereof, according to an embodiment.

FIG. 13 is a cross-sectional schematic of a portion of a fluid collection assembly that includes a porous material that is configured to minimize a decrease in the volume of the porous material when a length of the fluid collection assembly decreases, according to an embodiment.

FIG. 14 is a block diagram of a system for fluid collection, according to an embodiment.

### DETAILED DESCRIPTION

Embodiments are directed to fluid collection assemblies including one or more leak prevention features, systems including the same, and methods of using the same are disclosed herein. An example fluid collection assembly includes a fluid impermeable barrier at least defining a chamber, at least one opening, and a fluid outlet. The fluid collection assembly also include at least one porous material disposed in the chamber and one or more leak prevention features. The leak prevention features may include at least one of a shape of the fluid collection assembly that improves fit between the fluid collection assembly and a patient (e.g., an individual using the fluid collection assembly), at least one absorbent material at least partially disposed outside of the chamber, a base configured to have a patient disposed thereon, or one or more feature that allows a length of the fluid collection assembly to change.

During use, the fluid collection assembly is positioned adjacent to a urethral opening (e.g., vagina) of the patient. One or more bodily fluids (e.g., urine, blood, etc.) that are discharged from the urethral opening of the patient flow through the opening and into the chamber. The bodily fluids that enter the chamber are received into the porous material and are directed towards the fluid outlet. The bodily fluids may be removed from the chamber through the fluid outlet, for example, when a suction force from a vacuum source is applied to the fluid outlet.

Conventional fluid collection assemblies are prone to leaking (i.e., some of the bodily fluids fail to be received by the fluid collection assembly). Such leaking may cause unsanitary conditions, require clean-up, are uncomfortable, and may cause embarrassment for the patient. Conventional fluid collection assemblies are prone to leaking because the conventional fluid collection assemblies may form gaps between the region around the urethral opening and the conventional fluid collection assembly. The bodily fluids discharged from the urethral opening may leak through such gaps. The gaps may be formed, for example, because the conventional fluid collection assemblies have poor fit with the anatomy of the patient about the urethral opening, the conventional fluid collection assemblies need to contact the thighs of the patient to remain pressed against the urethral opening, and because the size of the region about the urethral opening may vary from patient to patient. Further, conventional fluid collection assemblies do not include a device that is configured to receive bodily fluids that fail to enter the chamber before such bodily fluids create unsanitary conditions, require clean-up, increase patient discomfort, and create embarrassment for the patient.

The fluid collection assemblies disclosed herein include one or more leak prevention features that are improvement over such conventional fluid collection assemblies. In an example, the leak prevention features may include a fluid collection assembly exhibiting one or more shapes that allow for better fit between the fluid collection assemblies disclosed herein and the patient (e.g., the vaginal region of the patient) than at least some conventional fluid collection assemblies. In an example, the leak prevention features may include one or more devices configured to collect bodily fluids that fail to enter the chamber before such bodily fluids may cause one or more create of unsanitary conditions, require clean-up, increase patient discomfort, or create embarrassment for the patient. In an example, the leak prevention features include a base that is configured to have a patient disposed thereon which may facilitate placement of the opening adjacent to the urethral opening. In an example, the leak prevention features include one or more features that allows the fluid collection assembly to change a shape thereof. In an example, the leak prevent features may include a combination of the leak prevent features disclosed herein.

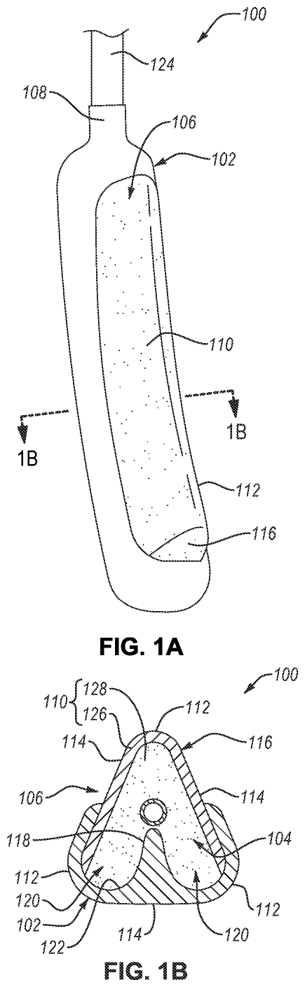

FIG. 1A is an isometric view of a fluid collection assembly 100, according to an embodiment. FIG. 1B is a cross-sectional schematic of the fluid collection assembly 100 taken along plane 1B-1B as shown in FIG. 1A. The fluid collection assembly 100 includes a fluid impermeable barrier 102. The fluid impermeable barrier 102 at least defines a chamber 104, at least one opening 106, and a fluid outlet 108. The fluid collection assembly 100 also includes at least one porous material 110 disposed in the chamber 104. The at least one porous material 110 may extend across the opening 106.

The fluid collection assembly 100 includes one or more leak prevention features. In an embodiment, as best shown in FIG. 1B, the one or more leak prevent features include the fluid collection assembly 100 exhibiting a generally triangular cross-sectional shape that includes three apexes 112 and three edges 114 extending between each of the apexes 112. The porous material 110 includes a protrusion 116 that extends outwardly from the opening 106. The protrusion 116 extends outwardly from the opening 106 (e.g., from at least a portion of the fluid impermeable barrier 102) at about 5 mm or more, about 1 cm or more, about 1.5 cm or more, about 2 cm or more, about 2.5 cm or more, about 3 cm or more, about 3.5 cm or more, about 4 cm or more, about 5 cm or more, or in ranges of about 5 mm to about 1.5 cm, about 1 cm to about 2 cm, about 1.5 cm to about 2.5 cm, about 2 cm to about 3 cm, about 2.5 cm to about 3.5 cm, about 3 cm to about 4 cm, or about 3.5 cm to about 5 cm. The protrusion 116 may include one of the apexes 112 and a portion of two of the edges 114 extending from (e.g., adjacent to) the apex 112.

Due to the shape of the protrusion 116 of the porous material 110, the fluid collection assembly 100 exhibits better fit against the region about the urethral opening (e.g., vaginal region) than if the fluid collection assembly exhibited a generally circular cross-sectional shape. For example, at least a portion of the protrusion 116 is able to be positioned between the labia folds (e.g., at least one of the labia major or the labia minor) due to the shape thereof. Positioning at least a portion of the protrusion 116 between the labia folds may prevent the fluid collection assembly 100 from shifting during use, especially shifting side-to-side. Preventing the fluid collection assembly 100 from shifting may prevent or inhibit the formation of gaps between the fluid collection assembly 100 and the patient. Further, positioning at least a portion of the protrusion 116 between the labia folds also may also position the porous material 110 closer to the urethral opening than if the porous material 110 exhibited a generally circular cross-sectional shape. Positioning the porous material 110 closer to the urethral opening causes a greater percentage of the bodily fluids that are discharged from the urethral opening to be received by the porous material 110 and directed into the chamber 104 thereby decreasing the quantity of bodily fluids that may leak than if the porous material 110 exhibited a generally circular cross-sectional shape.

In an embodiment, one or more of the apexes 112 are rounded which may prevent or inhibit the apexes 112 from pressing uncomfortably into the patient. The apexes 112 are rounded when the one or more of the apexes 112 exhibit an average radius of curvature that is about 0.5 mm or greater, about 1 mm or greater, about 2.5 mm or greater, about 5 mm or greater, about 7.5 mm or greater, about 1 cm or greater, about 1.5 cm or greater, about 2 cm or greater, or in ranges of about 0.5 mm to about 2.5 mm, about 1 mm to about 5 mm, about 2.5 mm to about 7.5 mm, about 5 mm to about 1 mm, about 7.5 mm to about 1.5 cm, or about 1 cm to about 2 cm. Generally, increasing the average radius of curvature of the apexes 112 makes using the fluid collection assembly 100 more comfortable. In an embodiment, one or more of the apexes 112 are not rounded (e.g., exhibits an average radius of curvature that is less than 0.5 mm). In such an embodiment, the portion of the fluid collection assembly 100 defining the one or more apexes 112 that are not rounded are sufficiently flexible that the apex 112 at least partially collapses when pressed into the patient to minimize patient discomfort. In an embodiment, at least one of the apexes 112 may exhibit an average radius of curvature that is different than another apex 112. For instance, the apex 112 formed by the protrusion 116 may exhibit an average radius of curvature that is less than the apexes 112 defined by the fluid impermeable barrier 102. The smaller average radius of curvature of the apex 112 defined by the protrusion 116 may facilitate insertion of the protrusion 116 between the labia folds and may position the porous material 110 closer to the urethral opening than if the apex 112 defined by the protrusion 116 exhibited a larger average radius of curvature. The larger radius of curvature of the apexes 112 defined by the fluid impermeable barrier 102 may minimize discomfort caused by such apexes 112 pressing into the thighs of the patient.

As previously discussed, the fluid collection assembly 100 includes a fluid impermeable barrier 102. The fluid impermeable barrier 102 may be formed of any suitable fluid imporous material(s), such as a fluid impermeable polymer (e.g., silicone, polypropylene, polyethylene, polyethylene terephthalate, a polycarbonate, etc.), a metal film, natural rubber, another suitable material, or combinations thereof. The fluid impermeable barrier 102 substantially prevents the bodily fluids from passing through the fluid impermeable barrier 102. In an example, the fluid impermeable barrier 102 may be air permeable and fluid impermeable. In such an example, the fluid impermeable barrier 102 may be formed of a hydrophobic material that defines a plurality of pores. At least a surface of the fluid impermeable barrier 102 that may contact the patient may be formed from a soft and/or smooth material (e.g., silicone), thereby reducing chaffing. In an embodiment, the fluid impermeable barrier 102 may be formed from a flexible material, such as silicone, which allows the fluid impermeable barrier 102 to be bent into a shape that conforms the anatomy of the patient.

In some examples, the fluid impermeable barrier 102 may be tubular (ignoring the opening 106), such as prismatic. During use, the outer surface of the fluid impermeable barrier 102 may contact the patient. The fluid impermeable barrier 102 may be sized and shaped to fit in the gluteal cleft between the legs of a female user.

The fluid impermeable barrier 102 may define at least one ridge 118 extending inwardly (e.g., into the chamber 104) from an interior surface 122 of the fluid impermeable barrier 102. The ridge 118 may extend inwardly from a portion of the fluid impermeable barrier 102 that forms the edge 114 opposite the protrusion 116 (e.g., opposite the opening 106). The ridge 118 may form a plurality of channels 120 in the chamber 104 through which the bodily fluids may flow. For instance, the channels 120 may be partially defined by the ridge 118 and adjacent portions of the fluid impermeable barrier 102. In an example, when the at least one ridge 118 includes a single ridge 118 (as illustrated), the single ridge 118 partially defines two channels 120. In an example, when the at least one ridge 118 includes a plurality of ridges, the plurality of ridges partially define three or more channels. It is currently believed that the channels 120 improve fluid flow through the chamber 104 such that more bodily fluids are removed from the chamber 104 than if the fluid impermeable barrier 102 did not include the channels 120.

The opening 106 provides an ingress route for fluids to enter the chamber 104. The opening 106 may be defined by the fluid impermeable barrier 102 such as by an inner edge of the fluid impermeable barrier 102. For example, the opening 106 is formed in and extends through the fluid impermeable barrier 102, from the proximal surface to an interior surface of the fluid impermeable barrier 102, thereby enabling bodily fluids to enter the chamber 104 from outside of the fluid collection assembly 100. The opening 106 may be an elongated hole in the fluid impermeable barrier 102. For example, the opening 106 may be defined as a cut-out in the fluid impermeable barrier 102. The opening 106 may be located and shaped to be positioned adjacent to a female urethral opening.

The fluid collection assembly 100 may be positioned proximate to the female urethral opening and urine or other bodily fluids may enter the chamber of the fluid collection assembly 100 via the opening 106. The fluid collection assembly 100 is configured to receive the bodily fluids into the chamber 104 via the opening 106. When in use, the opening 106 may have an elongated shape that extends from a first location below the urethral opening (e.g., at or near the anus or the vaginal opening) to a second location above the urethral opening (e.g., at or near the top of the vaginal opening or the pubic hair).

The opening 106 may have an elongated shape because the space between the legs of a female is relatively small when the legs of the female are closed, thereby only permitting the flow of the bodily fluids along a path that corresponds to the elongated shape of the opening 106 (e.g., longitudinally extending opening). The opening 106 in the fluid impermeable barrier 102 may exhibit a length measured along the longitudinal axis of the fluid collection assembly 100 that may be at least about 10% of the length of the fluid collection assembly 100, such as about 25% to about 50%, about 40% to about 60%, about 50% to about 75%, about 65% to about 85%, or about 75% to about 95% of the length of the fluid collection assembly 100.

The opening 106 in the fluid impermeable barrier 102 may exhibit a width measured transverse to the longitudinal axis of the fluid collection assembly 100 that may be at least about 10% of the circumference of the fluid collection assembly 100, such as about 25% to about 50%, about 40% to about 60%, about 50% to about 75%, about 65% to about 85%, or about 75% to about 100% of the circumference of the fluid collection assembly 100. The opening 106 may exhibit a width that is greater than 50% of the circumference of the fluid collection assembly 100 since the vacuum (e.g., suction) through the conduit 124 pulls the fluid through the porous material 110 and into the conduit 124.

In some examples, the opening 106 may be vertically oriented (e.g., having a major axis parallel to the longitudinal axis of the fluid collection assembly 100). In some examples (not shown), the opening 106 may be horizontally oriented (e.g., having a major axis perpendicular to the longitudinal axis of the fluid collection assembly 100). In an example, the fluid impermeable barrier 102 may be configured to be attached to the patient, such as adhesively attached (e.g., with a hydrogel adhesive) to the patient. According to an example, a suitable adhesive is a hydrogel layer.

As previously discussed, the fluid impermeable barrier 102 may define fluid outlet 108 configured to remove bodily fluids from the chamber 104. In some examples, the fluid outlet 108 is sized to receive the conduit 124. The conduit 124 may be disposed in the chamber 104 via the fluid outlet 108. The fluid outlet 108 may be sized and shaped to form an at least substantially fluid tight seal against the conduit 124 or the at least one tube substantially preventing the bodily fluids from escaping the chamber 104.

The fluid impermeable barrier 102 may include markings thereon, such as one or more markings to aid a user in aligning the fluid collection assembly 100 on the patient. For example, a line on the fluid impermeable barrier 102 (e.g., opposite the opening 106) may allow a healthcare professional to align the opening 106 over the urethral opening of the patient. In examples, the markings may include one or more of alignment guide or an orientation indicator, such as a stripe or hashes. Such markings may be positioned to align the fluid collection assembly 100 to one or more anatomical features such as a pubic bone, etc.

As previously discussed, the fluid collection assembly 100 includes porous material 110 disposed in the chamber 104. The porous material 110 may cover at least a portion (e.g., all) of the opening 106. The porous material 110 is exposed to the environment outside of the chamber 104 through the opening 106. The permeable properties referred to herein may be wicking, capillary action, absorption, diffusion, or other similar properties or processes, and are referred to herein as “permeable” and/or “porous.” The porous material 110 may also wick the bodily fluids generally towards an interior of the chamber 104, as discussed in more detail below. The porous material 110 may include one or more of a fluid permeable membrane 126 or a fluid permeable support 128.

In an embodiment, at least a portion of the porous material 110 may be a wicking material configured to wick the bodily fluids away from the opening 106, thereby preventing bodily fluids from escaping the chamber 104. The porous material 110 may not include absorption of the bodily fluids into the porous material 110. Put another way, substantially no absorption of the bodily fluids into the porous material 110 may take place after the wicking material is exposed to the bodily fluids. While no absorption is desired, the term “substantially no absorption” may allow for nominal amounts of absorption of the bodily fluids into the porous material 110 (e.g., absorbency), such as about 30 wt % of the dry weight of the porous material 110, about 20 wt %, about 15 wt %, about 10 wt %, about 7 wt %, about 5 wt %, about 3 wt %, about 2 wt %, about 1 wt %, or about 0.5 wt % of the dry weight of the porous material 110. In an embodiment, the porous material 110 may be at least one of an absorbent material or adsorbent material instead of or in addition to being a wicking material.

The fluid collection assembly 100 may include the fluid permeable membrane 126 disposed in the chamber 104. The fluid permeable membrane 126 may cover at least a portion (e.g., all) of the opening 106. The fluid permeable membrane 126 may be composed to pull/push the bodily fluids away from the opening 106, thereby promoting fluid flow into the chamber 104, prevent fluid remaining on the vulva of the patient, and preventing the bodily fluids from escaping the chamber 104.

The fluid permeable membrane 126 may include any material that may be permeable to the bodily fluids. For example, the fluid permeable membrane 126 may include fabric, such as a gauze (e.g., a silk, linen, or cotton gauze), another soft fabric, or another smooth fabric. Forming the fluid permeable membrane 126 from gauze, soft fabric, and/or smooth fabric may reduce chaffing caused by the fluid collection assembly 100 and makes wearing the fluid collection assembly more comfortable. In an embodiment, the fluid permeable membrane 126 may define a plurality of perforations or may be continuous (e.g., does not define perforations).

The fluid collection assembly 100 may include the fluid permeable support 128 disposed in the chamber 104. The fluid permeable support 128 is configured to support the fluid permeable membrane 126 and maintain the shape of the chamber 104 since the fluid impermeable barrier 102 and the fluid permeable membrane 126 may be formed from a relatively foldable, flimsy, or otherwise easily deformable material. For example, the fluid permeable support 128 may be positioned so the fluid permeable membrane 126 is disposed between the fluid permeable support 128 and the fluid impermeable barrier 102. The fluid permeable support 128 may support and maintain the position of the fluid permeable membrane 126 and the shape of the chamber 104. The fluid permeable support 128 may include any material that may be permeable to the bodily fluids, such as any of the fluid permeable membrane 126 materials disclosed above. For example, the fluid permeable membrane 126 material(s) may be utilized in a more dense or rigid form than in the fluid permeable membrane 126 when used as the fluid permeable support 128. The fluid permeable support 128 may be formed from any fluid porous material that is less deformable than the fluid permeable membrane 126. For example, the fluid permeable support 128 may include a porous polymer (e.g., nylon, polyester, polyurethane, polyethylene, polypropylene, etc.) structure (e.g., spun fibers such as spun nylon fibers) or a foam (e.g., an open cell foam). In some examples, the fluid permeable support 128 may be formed from a natural material, such as cotton, wool, silk, or combinations thereof. In such examples, the material may have a coating to prevent or limit absorption of the bodily fluids into the material, such as a water repellent coating. In some examples, the fluid permeable support 128 may be formed from fabric, felt, gauze, or combinations thereof.

In some examples, the fluid permeable membrane 126 may be optional. For example, the porous material 110 may include only the fluid permeable support 128. In some examples, the fluid permeable support 128 may be optionally omitted from the fluid collection assembly 100 and the porous material 110 may only include the fluid permeable membrane 126.

In an embodiment, the fluid permeable membrane 126 and/or the fluid permeable support 128 are wicking materials. In such an embodiment, the fluid permeable support 128 may have a greater ability to wick the bodily fluids than the fluid permeable membrane 126. In some examples, the wicking ability of the fluid permeable support 128 and the fluid permeable membrane 126 may be substantially the same. In an embodiment, the fluid permeable membrane 126 and/or the fluid permeable support 128 are non-wicking materials (e.g., absorbent and/or adsorbent materials).

In an embodiment, the fluid permeable membrane 126 and the fluid permeable support 128 may at least substantially completely fill the portions of the chamber 104 not occupied by the conduit 124. In an embodiment, the fluid permeable membrane 126 and the fluid permeable support 128 may not substantially completely fill the portions of the chamber 104 not occupied by the conduit 124. In such an embodiment, the fluid collection assembly 100 includes the fluid reservoir disposed in the chamber 104.

The fluid reservoir is a substantially unoccupied portion of the chamber 104. The fluid reservoir may be defined between the fluid impermeable barrier 102 and at least one of the fluid permeable membrane 126 or the fluid permeable support 128. The bodily fluids in the chamber 104 may flow through the fluid permeable membrane 126 and/or fluid permeable support 128 to the fluid reservoir. The fluid reservoir may retain of the bodily fluids. The bodily fluids in the chamber 104 may flow through the fluid permeable membrane 126 and/or fluid permeable support 128 and, optionally, to the fluid reservoir. The fluid impermeable barrier 102 may retain the bodily fluids in the fluid reservoir. The fluid reservoir may be in a portion of the chamber 104 designed to be in a gravimetrically low point of the fluid collection assembly 100 when the fluid collection assembly 100 is worn.

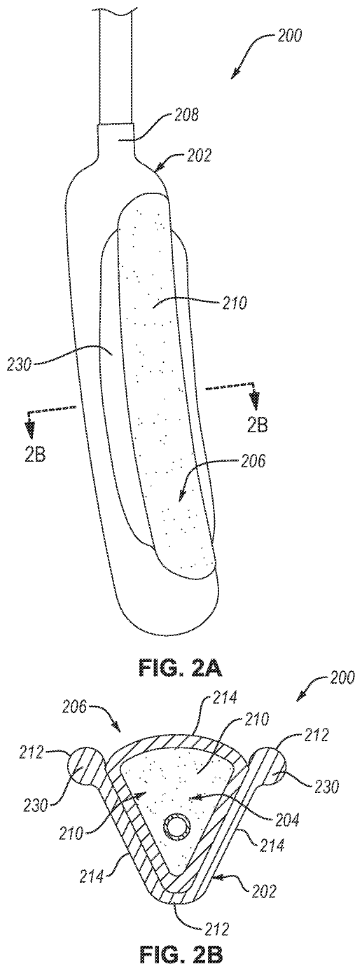

FIG. 2A is an isometric view of a fluid collection assembly 200, according to an embodiment. FIG. 2B is a cross-sectional schematic of the fluid collection assembly 200 taken along plane 2B-2B as shown in FIG. 2A. Except as otherwise disclosed herein, the fluid collection assembly 200 is the same or substantially similar to any of the fluid collection assemblies disclosed herein and may include any of the features of any of the fluid collection assemblies disclosed herein. For example, the fluid collection assembly 200 may include a fluid impermeable barrier 202 defining a chamber 204, at least one opening 206, and a fluid outlet 208. The fluid collection assembly 200 may also include at least one porous material 210 disposed in the chamber 204.

The fluid collection assembly 200 exhibits a generally triangular cross-sectional shape that includes three apexes 212 and three edges 214 extending between the apexes 212. The porous material 210 includes a portion extending across the opening 206. The portion of the porous material 210 that extends across the opening 206 defines an edges 214 of the fluid collection assembly 200. The portion of the porous material 210 that extends across the opening 206 may also extend outwardly from the opening 206. When the porous material 210 extends outwardly from the opening 206, the porous material 210 may form a protrusion that includes two of the apexes 212. The fluid impermeable barrier 202 defines the apex 212 that is opposite the opening 206 and at least a portion of the edges 214 extending from the apex 212 formed by the fluid impermeable barrier 202 extending towards the other apexes 212 (e.g., towards the opening 206). In an embodiment, the porous material 210 does not extend outwardly from the opening 206. In an embodiment, the porous material 210 extends outwardly from the opening 206 to form a protrusion.

The fluid collection assembly 200 is configured to be positioned between the labia folds. For example, the labia folds may be spread apart and the fluid collection assembly 200 may be positioned between the spread apart labia folds such that the opening 206 faces the urethral opening. The labia folds may be released after positioning the fluid collection assembly 200. The labia folds may then clamp around a portion of the fluid collection assembly 200 which may help keep the fluid collection assembly 200 positioned against the urethral opening thereby minimizing the likelihood that bodily fluids discharged from the urethral opening leak.

The fluid impermeable barrier 202 may define one or more nubs 230. The nubs 230 are regions of the fluid impermeable barrier 202 exhibiting greater thickness than the areas thereabout and may, for example, exhibit a generally circular cross-section (as shown in FIG. 2B). The nubs 230 may be located at the portion of the fluid impermeable barrier 202 that defines the opening 206 or at another location that is likely to contact the labia folds. The nubs 230 may prevent the fluid impermeable barrier 202 from uncomfortably pressing into the labia folds.

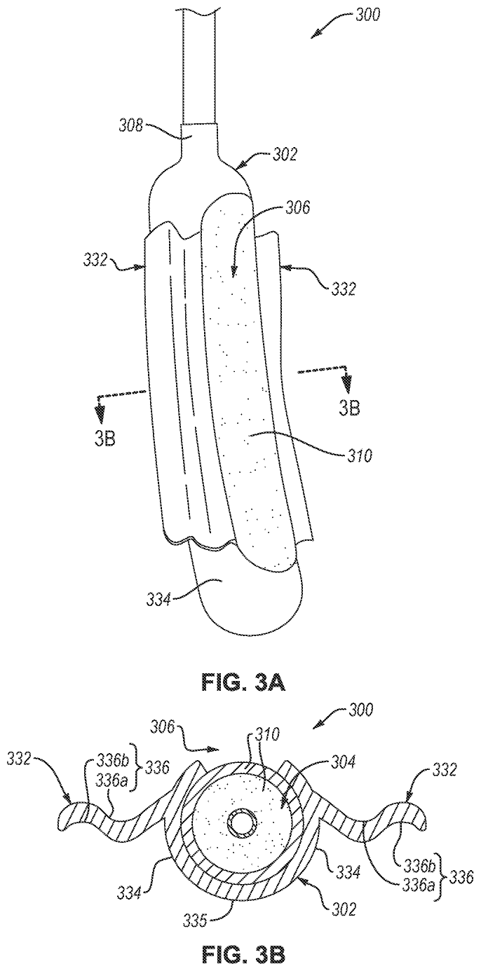

FIG. 3A is an isometric view of a fluid collection assembly 300, according to an embodiment. FIG. 3B is a cross-sectional schematic of the fluid collection assembly 300 taken along plane 3B-3B as shown in FIG. 3A. Except as otherwise disclosed herein, the fluid collection assembly 300 is the same or substantially similar to any of the fluid collection assemblies disclosed herein and may include any of the features of any of the fluid collection assemblies disclosed herein. For example, the fluid collection assembly 300 may include a fluid impermeable barrier 302 defining a chamber 304, at least one opening 306, and a fluid outlet 308. The fluid collection assembly 300 may also include at least one porous material 310 disposed in the chamber 304.

The fluid collection assembly 300 includes two wings 332 extending from the fluid impermeable barrier 302. The each of the two wings 332 may extend from a portion of a lateral surface 334 of the fluid impermeable barrier 302. For example, the two wings 332 may extend from a portion of the lateral surface 334 that defines the opening 306 or is closer to the opening 306 than a back surface 335 of the fluid impermeable barrier 302 (e.g., a surface of the fluid impermeable barrier 302 that is opposite the opening 306). In an embodiment, the two wings 332 may be integrally formed with the fluid impermeable barrier 302.

The two wings 332 form the leak prevention feature of the fluid collection assembly 300 since the wings 332 are configured to contact the thighs of the individual during use. For example, as previously discussed, convention fluid collection assemblies rely on contact between the conventional fluid collection assemblies and the thighs of the patient to maintain the position and shape (e.g., bent shape) of the conventional fluid collection assemblies to limit leaks. However, movement of the patient and/or thin thighs may prevent the conventional fluid collection assemblies from maintaining contact against the thighs of the patient. The two wings 332 of the fluid collection assembly 300 may maintain contact between the thighs of the patient and the fluid collection assembly 300 even when the patient moves or has thin thighs. In particular, the two wings 332 increase the effective width of the fluid collection assembly 300 thereby allowing the fluid collection assembly 300 to maintain contact with the thighs of the patient. For example, the two wings 332 allow the fluid collection assembly 300 to maintain contact with the thighs even when the spacing between the thighs is greater than a maximum width of the fluid collection assembly 300 measured between the lateral surfaces 334 of the fluid impermeable barrier 302. The contact between the two wings 332 and the thighs may be sufficient to at least one of maintain the position of the fluid collection assembly 300 (e.g., maintain the opening 306 adjacent to the urethral opening) or maintain the shape of the fluid collection assembly 300 (e.g., the fluid collection assembly 300 is bent to general correspond to the shape of the region about the urethral opening). Thus, the two wings 332 minimize the likelihood that bodily fluids leak from the fluid collection assembly 300 since the two wings 332 improve contact between the thighs of the patient and the fluid collection assembly 300.

The two wings 332 may act as a spring which may minimize patient discomfort. Configuring the two wings 332 to act as a spring allows the two wings 332 to flex and bend as the space between the thighs of the patient change while still being able to maintain contact with the patient. For example, when the space between the thighs of the patient decrease, the two wings 332 may be configured to flex. Flexing the two wings 332 may minimize the amount that the two wings 332 press into the thighs of the patient thereby making the fluid collection assembly 300 more comfortable than if the two wings 332 did not flex. Further, flexing the two wings 332 may decrease a compressive force applied from the wings 332 to the fluid impermeable barrier 302 that decreases a volume of the chamber 304. Further, increasing the distance between the thighs of the individual allows the wings 332 to at least partially return to their original shape such that the wings 332 maintain contact with the thighs of the patient.

In an embodiment, the two wings 332 may include one or more curves 336 formed therein. The curves 336 may allow the two wings 332 to act as a spring since the curves 336 provide preferential locations for the two wings 332 to bend (e.g., the curves 336 allow the wings 332 to act similar to leaf springs). The curves 336 may be concave or convex relative to the opening 306. In an example, at least one of the curves 336 is concave relative to the opening 306. The concave curves 336 form fluid channels in which bodily fluids that leak from the chamber 304 may flow. The concave curves 336 may be positioned adjacent to the opening 306 which may encourage the leaked bodily fluids to reenter the chamber 304 through the opening 306.

In an example, each of the two wings 332 may include a plurality of curves 336. The plurality of curves 336 provide a plurality of locations at which the wings 332 may flex. In an embodiment, as illustrated, the plurality of curves 336 includes a first curve 336a and a second curve 336b. One of the first curve 336a or the second curve 336b may be concavely curved relative to the opening 306 while the other of the first curve 336a or the second curve 336b is convexly curved relative to the opening 306. The different direction of the curvature of the first and second curves 336a, 336b allows the wings 332 to flex even when one side of the wings 332 is obstructed (e.g., by the thighs) such that one of the first curve 336a or the second curve 336b cannot flex.

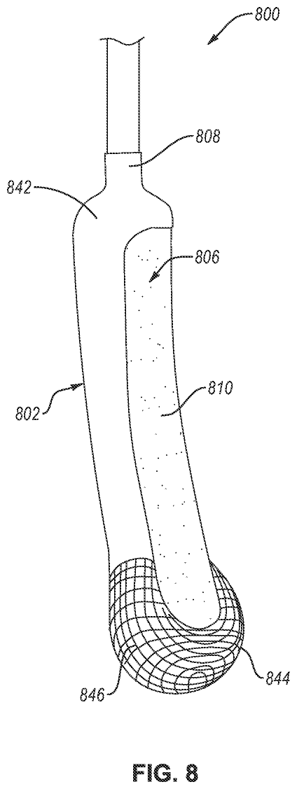

The wings 332 may be formed of a fluid impermeable material, such as any of the fluid impermeable materials disclosed herein. As such, the wings 332 may form channels in which the bodily fluids may flow. The channels may at least one direct the bodily fluids back into the chamber 306, direct the bodily fluids to an absorbent material (e.g., absorbent element 846 shown in FIG. 8), or limit bodily fluids to a limited region of the patient. In an example, the wings 332 may be formed from the same material as the fluid impermeable barrier 302. In such an example, the wings 332 may be separately formed from and attached to the fluid impermeable barrier 302 or integrally formed with the fluid impermeable barrier 302 (e.g., the fluid impermeable barrier 302 and the wings 332 exhibit single piece construction). In an example, the wings 332 may be formed from a material that is different than the fluid impermeable barrier 302.

In an embodiment, the wings 332 may include one or more reinforcement features that are configured to increase the rigidity of the wings 332. For example, the wings 332 may be formed from a material (e.g., silicone) that is unable to maintain the shape thereof when unsupported which may cause the wings 332 to sag. Wings 332 that sag are unable to act as a spring and may be unable to maintain contact with the thighs of the patient. The reinforcement features may include a coating, wire, mesh, or other structure applied or incorporated into the wings 332 that increase the rigidity of the wings 332 such that the wings 332 may generally maintain the shape thereof when unsupported. The reinforcement features may be formed from a material exhibiting a Young's modulus (i.e., elastic modulus) that is greater than the material that forms the bulk of the wings 332. For instance, the reinforcement features may be formed of a metal (e.g., steel, aluminum, or copper) while the bulk of the wings 332 are formed from silicone or other polymer.

In an example, as illustrated, the fluid impermeable barrier 302 may be substantially cylindrical. In another example, the fluid impermeable barrier 302 may exhibit a non-cylindrical shape, such as an oblong, prismatic, flattened tubes, or any of the other shapes disclosed herein.

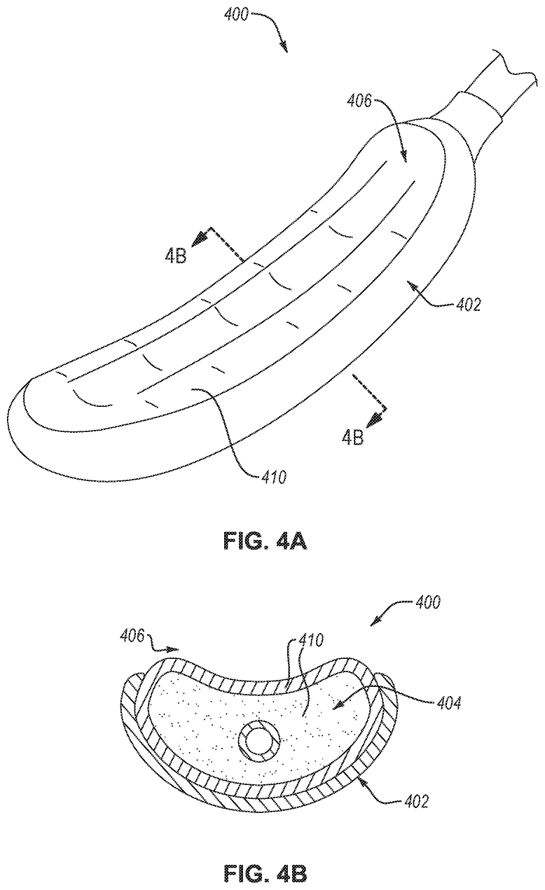

FIG. 4A is an isometric view of a fluid collection assembly 400, according to an embodiment. FIG. 4B is a cross-sectional schematic of the fluid collection assembly 400 taken along plane 4B-4B as shown in FIG. 4A. Except as otherwise disclosed herein, the fluid collection assembly 400 is the same or substantially similar to any of the fluid collection assemblies disclosed herein and may include any of the features of any of the fluid collection assemblies disclosed herein. For example, the fluid collection assembly 400 may include a fluid impermeable barrier 402 at least defining an chamber 404 and an opening 406. The fluid collection assembly 400 may also include at least one porous material 410 disposed in the chamber 404.

The portion of the porous material 410 that extends across the opening 406 may be concavely curved relative to an exterior of the fluid collection assembly 400. The concave curvature of the porous material 410 may form a leak prevention feature of the fluid collection assembly 400. In an example, the concave curvature of the portion of the fluid collection assembly 400 may be configured to receive at least a portion the labia folds there within. Thus, the labia folds are positioned within the chamber 404 and any fluids that flow through the labia folds are already in the chamber 404. In an example, the labia folds are configured to be spread apart and the fluid collection assembly 400 is positioned between the labia folds, similar to what was discussed with regards to FIGS. 2A and 2B. In such an example, the concave curvature of the portion of the porous material 410 may facilitate the collection of bodily fluids that are discharged from the urethral opening. For instance, the concave curvature may be configured to accommodate anatomical features between the labia folds (e.g., the labia minor if only the labia major are spread apart) and/or the concavity of the porous material 410 may facilitate the capture of bodily fluids when discharged at oblique angles from the urethral opening.

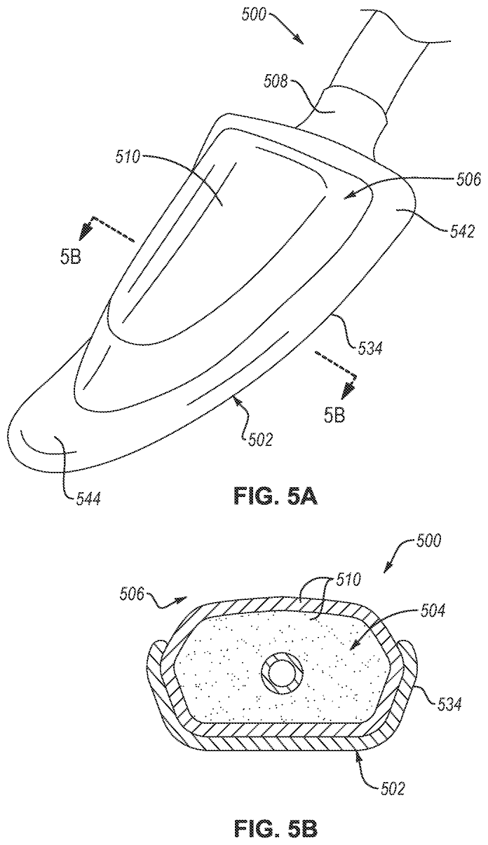

FIG. 5A is an isometric view of a fluid collection assembly 500, according to an embodiment. FIG. 5B is a cross-sectional schematic of the fluid collection assembly 500 taken along plane 5B-5B as shown in FIG. 5A. Except as otherwise disclosed herein, the fluid collection assembly 500 is the same or substantially similar to any of the fluid collection assemblies disclosed herein and may include any of the features of any of the fluid collection assemblies disclosed herein. For example, the fluid collection assembly 500 may include a fluid impermeable barrier 502 at least defining an chamber 504, an opening 506, and a fluid outlet 508. The fluid collection assembly 500 may also include at least one porous material 510 disposed in the chamber 504.

The fluid collection assembly 500 includes a proximal end region 542 that (as shown) defines the fluid outlet 508 and a distal end region 544 opposite the proximal end region 542. The fluid impermeable barrier 502 exhibits a tapered shape from the proximal end region 542 to the distal end region 544. For example, the fluid impermeable barrier 502 exhibits a width measured perpendicularly to a longitudinal axis thereof between the lateral surfaces 534 thereof. The width of the fluid impermeable barrier 502 is greatest at or near the proximal end region 542. The width of the fluid impermeable barrier 502 decreases from the greatest width to the distal end region 544. The tapered shape of the fluid impermeable barrier 502 may generally correspond to the tapered space between the thighs thereby allowing the fluid collection assembly 500 to be positioned between the thighs with the widest portion of the fluid impermeable barrier 502 adjacent to the mons pubis.

The tapered shape of the fluid impermeable barrier 502 is a leak prevent feature. For example, the tapered shape of the fluid impermeable barrier 502 also allows the opening 506 to exhibit a generally tapered shape. The tapered shape of the opening 506 allows the width of the opening 506 at or near the urethral opening to be wider than if the fluid impermeable barrier 502 exhibited a non-tapered shape. The wider opening 506 may minimize the amount of bodily fluids discharged from the urethral opening that did not enter the chamber 504 through the opening 506. Further, the amount of bodily fluids that enter the chamber 504 through the portions of the opening 506 near the distal end region 544 is limited and, thus, the narrowed width of the opening 506 near the distal end region 544 has a minimal effect on the quantity of bodily fluids that leak. In fact, the fluid impermeable barrier 502 may define a fluid reservoir (not shown) at or near the distal end region 544 and the minimized width of the opening 506 near the distal end region 544 may actually minimize bodily fluids that leak from the fluid reservoir.

In an embodiment, a portion of the porous material 510 may protrude from the opening 506. The protrusion of the porous material 510 may allow the porous material 510 to be positioned closer to the urethral opening of the patient. Thus, protruding portions of the porous material may be a leak prevention feature.

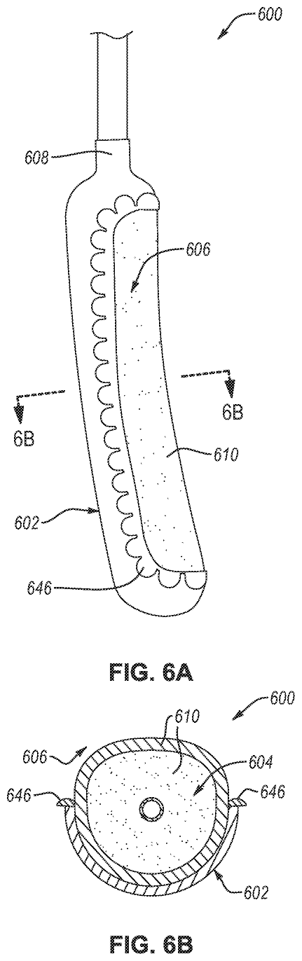

The fluid collection assemblies disclosed herein may include one or more absorbent elements that are configured to catch bodily fluids that would otherwise leak from fluid collection assembly, as discussed in more detail with regards to FIGS. 6A-9B. For example, FIG. 6A is an isometric view of a fluid collection assembly 600, according to an embodiment. FIG. 6B is a cross-sectional schematic of the fluid collection assembly 600 taken along plane 6B-6B as shown in FIG. 6A. Except as otherwise disclosed herein, the fluid collection assembly 600 may be the same or substantially similar to any of the fluid collection assemblies disclosed herein and may include any of the features of any of the fluid collection assemblies disclosed herein. For example, the fluid collection assembly 600 may include a fluid impermeable barrier 602 defining a chamber 604, an opening 606, and a fluid outlet 608. The fluid collection assembly 600 also includes at least one porous material 610 disposed in the chamber 604. The fluid collection assembly 600 may exhibit any of the shapes disclosed herein (e.g., cylindrical, as shown).

The fluid collection assembly 600 includes an absorbent element 646 that is configured to absorb bodily fluids adjacent or proximate to the opening 606. For example, the absorbent element 646 may be attached to or otherwise disposed on or near at least a portion of the fluid impermeable barrier 602 that defines the opening 606. The absorbent element 646 is a leak prevention feature. For example, bodily fluids that leak from the chamber 604 are likely to come in contact with the absorbent element 646 due to the proximity of the absorbent element 646 to the opening 606. The absorbent element 646 may receive and store therein at least some of the bodily fluids that come in contact with the absorbent element 646. Thus, the absorbent element 646 decreases the quantity of bodily fluids that leak from the fluid collection assembly 600.

The absorbent element 646 includes at least one absorbent material. The absorbent material may include any absorbent material that allows for absorption of a significant amounts of the bodily fluids. For example, the quantity of the bodily fluids that the absorbent element 646 may absorb may be about 10 wt % or greater, about 25 wt % or greater, about 50 wt % or greater, about 100 wt % or greater, about 250 wt % or greater, 500 wt % or greater, about 1000 wt % or greater, about 2,500 wt % or greater, about 5,000 wt % or greater, about 10,000 wt % or greater, about 50,000 wt % or greater, about 100,000 wt % or greater, or in ranges of about 10 wt % to about 50 wt %, about 25 wt % to about 100 wt %, about 50 wt % to about 250 wt %, about 100 wt % to about 500 wt %, about 250 wt % to about 1000 wt %, about 500 wt % to about 2,500 wt %, 1000 wt % to about 5,000 wt %, about 2,500 wt % to about 10,000 wt %, about 5,000 wt % to about wt %, about 10,000 wt % to about 100,000 wt % of the dry weight of the absorbent material. Examples of absorbent materials includes cotton, other fabrics, gauze, cellulose, polypropylene, polymer sponges, super absorbent polymers, or other absorbent materials. It is noted that, as used herein, absorbent also includes adsorbent.

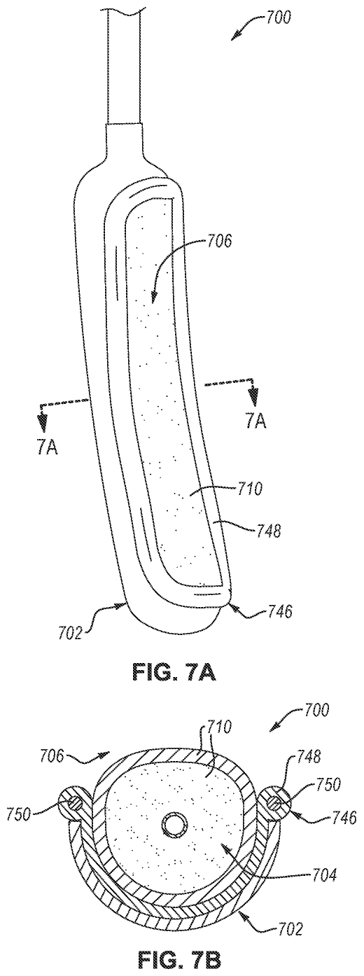

The absorbent element 646 may be attached to the fluid impermeable barrier 602 using any suitable technique. In an example, the absorbent element 646 may be adhesively or mechanically (e.g., clamped) attached to the fluid impermeable barrier 602. In an example, a portion of the absorbent element 646 may be disposed in the chamber 604, such as between the fluid impermeable barrier 602 and the porous material 610 (as shown in FIG. 7B). In such an example, friction between the absorbent element 646 and at least one of the fluid impermeable barrier 602 or the porous material 610 maintain the position of the absorbent element 646.

It is noted that the absorbent element 646 is likely to maintain the bodily fluids therein for as long as the fluid collection assembly 600 is used since the absorbent element 646 is not a wicking material. The constant moisture content of the absorbent element 646 after receiving the bodily fluids may cause patient discomfort and skin degradation. However, the absorbent element 646 may contain the discomfort and skin degradation to a minimal area unlike bodily fluids that leak from the fluid collection assembly 600 and, thus, the absorbent element 646 is an improvement over allowing the bodily fluids to leak. Further, the absorbent element 646 may be positioned immediately adjacent to and/or contact the porous material 610. Such positions of the absorbent element 646 may cause at least some of the bodily fluids received by the absorbent element 646 to flow into the porous material 610 and/or the air flow through the opening 606 may encourage evaporation of the bodily fluids received by the absorbent element 646 thereby inhibiting patient discomfort and skin degradation.

FIG. 7A is an isometric view of a fluid collection assembly 700, according to an embodiment. FIG. 7B is a cross-sectional schematic of the fluid collection assembly 700 taken along plane 7B-7B as shown in FIG. 7A. Except as otherwise disclosed herein, the fluid collection assembly 700 is the same or substantially similar to any of the fluid collection assemblies disclosed herein and may include any of the features of any of the fluid collection assemblies disclosed herein. For example, the fluid collection assembly 700 may include a fluid impermeable barrier 702 that at least defines a chamber 704 and at least one opening 706. The fluid collection assembly 700 may also include at least one porous material 710 disposed in the chamber 704.

The fluid collection assembly 700 includes at least one absorbent element 746. Except as otherwise disclosed herein, the absorbent element 746 may be the same or substantially similar to any of the absorbent elements 746 disclosed herein. For example, the absorbent element 746 may be positioned on or near the portions of the fluid impermeable barrier 702 that define the opening 706. The absorbent element 746 is configured to receive and store at least some of bodily fluids that would otherwise leak from the fluid collection assembly 700 if the fluid collection assembly 700 did not include the absorbent element 746. Thus, the absorbent element 746 is a leak prevention feature.

The absorbent element 746 may include a plurality of layers. For example, as illustrated, the absorbent element 746 may include a first layer 748 and a second layer 750. In an embodiment, the first layer 748 may form an external layer of the absorbent element 746 and the second layer 750 may be an interior layer. As such, the first layer 748 may contact the patient and the second layer 750 does not contact the patient when the fluid collection assembly 700 is being used (e.g., the second layer 750 is spaced from the patient by the first layer 748). Since the first layer 748 is configured to contact the patient, the first layer 748 may be configured to be more comfortable when contacting the patient than the second layer 750. In an example, the first layer 748 may be formed from a material that is smoother and/or softer than the second layer 750 to prevent chaffing of the patient. In an example, the first layer 748 may be formed from a wicking material or a less absorbent material than the second layer 750. In such an example, the bodily fluids that are received by the absorbent element 746 are predominately stored in the second layer 750 while the first layer 748 remains drier than the second layer 750. Thus, the absorbent element 746 may minimize the moisture that remains in contact with the skin of the patient thereby minimizing patient discomfort and/or skin degradation than if the first layer 748 exhibited the same or greater absorption than the second layer 750.

In a particular example, the second layer 750 may be formed of a super absorbent polymer (e.g., a polymer that can absorb at least about 5 times, at least about 10 times, at least about 30 times, at least about 50 times, or at least about 100 times its dry weight of bodily fluids) while the first layer 748 is formed from flexible material that is less absorbent than the second layer 750 (e.g., a wicking material and/or a gauze). In such an example, the second layer 750 may absorb sufficient quantities of bodily fluids that the volume thereof significantly increases. The increase in volume of the second layer 750 may cause the absorbent element 746 to conform to the shape of the region about the urethral opening of the patient which may inhibit or substantially prevent bodily fluids from leaking from the fluid collection assembly 700. For instance, portions of the second layer 750 that are adjacent to portions of the first layer 748 that are spaced from the patient may exhibit an increase in volume that is greater than portions of the second layer 750 that are adjacent to portions of the first layer 748 that do contact the patient. Thus, the volume increase of the second layer 750 may cause the absorbent element 746 to fill any gaps between the patient and the absorbent element 746. In some embodiments, the second layer 750 may be an external layer instead of or in addition to the first layer 748.

In an embodiment, a portion of the absorbent element 746 is positioned within the chamber 704 which may attach the absorbent element 746 to the fluid impermeable barrier 702. For example, as illustrated, a portion of the absorbent element 746 (e.g., the first layer 748) may extend between and separate at least a portion of the fluid impermeable barrier 702 from at least a portion of the porous material 710. In other words, the portion of the absorbent element 746 that is disposed in the chamber 704 may extend from a first portion of the absorbent element 746 on one side of the opening 706 to a second portion of the absorbent element 746 that is on an opposing side of the opening 706. In such an example, the fluid collection assembly 700 may be formed by positioning a portion of the absorbent element 746 in the chamber 704 and then positioning the porous material 710 in the chamber 704.

FIG. 8 is an isometric view of a fluid collection assembly 800, according to an embodiment. Except as otherwise disclosed herein, the fluid collection assembly 800 may be the same or substantially similar to any of the fluid collection assemblies disclosed herein and may include any of the features of any of the fluid collection assemblies disclosed herein. For example, the fluid collection assembly 800 may include fluid impermeable barrier 802 that forms a proximal end region 842 and a distal end region 844. The fluid impermeable barrier 802 may at least define a chamber (not shown, obscured), at least one opening 806, and a fluid outlet 808. The fluid outlet 808 may be at or near the proximal end region 842. The fluid collection assembly 800 may also include at least one porous material 810 disposed in the chamber.

During use, the distal end region 844 is position at or near the buttocks or perineum of the patient. Thus, the distal end region 844 generally forms the gravimetric low-point of the fluid collection assembly 800. Bodily fluids that leak from the fluid collection assembly 800 generally flow in the direction of gravity and flow towards the distal end region 844. The fluid collection assembly 800 may include at least one absorbent element 846 that covers at least a portion (e.g., all) of the distal end region 844. Except as otherwise disclosed herein, the absorbent element 846 may be the same or substantially similar to any of the absorbent elements disclosed herein. The absorbent element 846 may receive and store the bodily fluids that flow towards the distal end region 844 due to its position on the distal end region 844. Thus, the absorbent element 846 is a leak prevention feature. It is noted that the absorbent element 846 may also be positioned at or near at least a portion of the opening 806, as previously discussed herein.

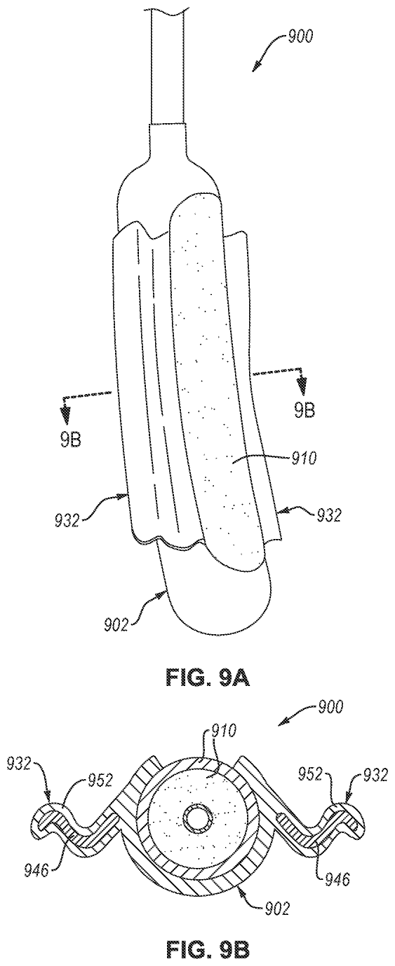

FIG. 9A is an isometric view of a fluid collection assembly 900, according to an embodiment. FIG. 9B is a cross-sectional schematic of the fluid collection assembly 900 shown in FIG. 9A taken along plane 9B-9B. Except as otherwise disclosed herein, the fluid collection assembly 900 is the same or substantially similar to any of the fluid collection assemblies disclosed herein and may include any of the features of any of the fluid collection assemblies disclosed herein. For example, the fluid collection assembly 900 may include a fluid impermeable barrier 902 and at least one porous material 910.

The fluid collection assembly 900 includes two or more wings 932. Except as otherwise disclosed herein, the two or more wings 932 are the same or substantially similar to the wings 332 shown in FIGS. 3A and 3B. The two wings 932 includes an absorbent element 946. The absorbent element 946 may receive and store at least some of the bodily fluids that reach the wings 932. Thus, the absorbent element 946 is a leak prevention feature.

In an embodiment, as illustrated, the wings 932 includes an outer layer 952 and the absorbent element 946 is disposed within the outer layer 952. For example, the outer layer 952 may define a cavity and the absorbent element 946 may be disposed in and at least partially occupy the cavity. The outer layer 952 is permeable to the bodily fluids such that the bodily fluids may reach the outer layer 952 and be absorbed by the absorbent element 946. The outer layer 952 is permeable to the bodily fluids when, for instance, the outer layer 952 is formed from at least one of an absorbent material, a wicking material, or a fluid impermeable material that defines a plurality holes therein.

In an embodiment, the absorbent element 946 includes a super absorbent polymer and the absorbent element 946 is enclosed by the outer layer 952. The volume of the super absorbent polymer may increase when the super absorbent polymer absorbs the bodily fluids. However, the outer layer 952 may apply a compressive force against the absorbent element 946 when the volume of the absorbent element 946 increases beyond the volume of the cavity defined by the outer layer 952. The compressive force may cause the absorbent element 946 to increase in rigidity as the absorbent element 946 continues to absorb more bodily fluids. The increased rigidity of the absorbent element 946 causes the absorbent element 946 to act similar to a reinforcement feature discussed above (e.g., allows the wings 932 to better maintain a shape thereof when unsupported) thereby allowing the wings 932 to press against the thighs better and limit bodily fluids flowing between the wings 932 and the thighs. For example, the quantity of bodily fluids that the absorbent element 946 needs to absorb to increase the rigidity thereof may indicate that the fluid collection assembly 900 is moving relative to the urethral opening and forming gaps between the fluid collection assembly 900 and the region about the urethral opening. However, the increased rigidity of the wings 932 may improve contact between the thighs of the patient and the fluid collection assembly 900 thereby inhibiting movement of the fluid collection assembly 900 relative to the urethral opening. In other words, the absorbent element 946 may allow the fluid collection assembly 900 to self-fix positioning issues when the absorbent element 946 includes a super absorbent polymer.

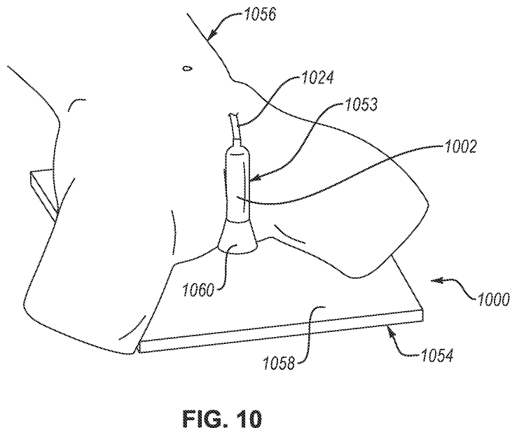

FIG. 10 is an isometric view of a fluid collection assembly 1000, according to an embodiment. The fluid collection assembly 1000 includes a fluid collection component 1053. The fluid collection component 1053 includes a fluid impermeable barrier 1002, at least one porous material (not shown, obscured) disposed in the fluid impermeable barrier 1002, and, optionally, a conduit 1024. Except as otherwise disclosed herein, the fluid collection component 1053 may be the same or substantially similar to any of the fluid collection assemblies disclosed herein (e.g., may include any of the features of any of the fluid collection assemblies disclosed herein) or any convention fluid collection assembly that includes a fluid impermeable barrier defining an opening and at least one porous material. The fluid collection assembly 1000 also includes a base 1054. The fluid collection component 1053 is secured, directly or indirectly, to the base 1054. A patient 1056 using the fluid collection assembly 1000 may sit, lie down on, or otherwise be positioned on the base 1054 such that the urethral opening of the patient 1056 is positioned adjacent to the opening defined by the fluid impermeable barrier 1002. In other words, the patient 1056 may be positioned on the base 1054 such that the urethral opening of the patient 1056 is adjacent to or, more preferably, presses against the porous material.

The base 1054 forms a leak prevention feature of the fluid collection assembly 1000. For example, the base 1054 may include a top surface 1058 that is configured to prevent the base 1054 from sliding when the patient 1056 is positioned on the base 1054. For example, the top surface 1058 may be textured, be formed by a material having a coefficient of static friction against dry skin of the patient 1056 that is sufficient to prevent the patient 1056 from sliding on the base 1054 (e.g., a coefficient of static friction that is greater than about 0.8, greater than about 1, or greater than about 1.5), or defines one or more divots and/or protrusions that are configured to receive or fit between anatomical features of the patient 1056 (e.g., two divots to receive the buttocks of the patient 1056 and/or a protrusion configured to fit between the buttocks of the patient 1056). The base 1054 also limits movement of the fluid collection component 1053 relative to the base 1054 thereby limiting movement of the fluid collection component 1053 relative to the patient 1056. Thus, the patient 1056 may be positioned on the base 1054 such that the urethral opening of the patient 1056 is adjacent to the opening and the base 1054 maintains the position of the opening adjacent to the urethral opening. Further, the base 1054 allows the patient 1056 to press against the fluid collection component 1053 such that there are substantially no gaps between the fluid collection component 1053 and the region about the urethral opening. The patient 1056 may move away from the fluid collection component 1053 for comfort reasons if the patient 1056 is uncomfortably pressing against the fluid collection component 1053.

The fluid collection component 1053 may be attached to the base 1054 using any suitable technique. In an embodiment, at least a portion fluid collection component 1053 (e.g., the fluid impermeable barrier 1002) is integrally formed with or otherwise permanently attached to (e.g., via an adhesive) to the base 1054. In an embodiment, the fluid collection component 1053 may be temporarily attached to the base 1054. In such an embodiment, the base 1054 may define a hole (not shown, occupied) extending at least partially therethrough. The hole is configured to receive the fluid collection component 1053. The hole may be configured to have the fluid collection component 1053 secured thereto, for example, via an interference fit. In an example, the base 1054 include a projection 1060 that defines the hole (e.g., the hole extends at least partially through the projection 1060). The projection 1060 may increase the surface area of the fluid collection component 1053 and the base 1054 that contact each other which may facilitate an interference fit.

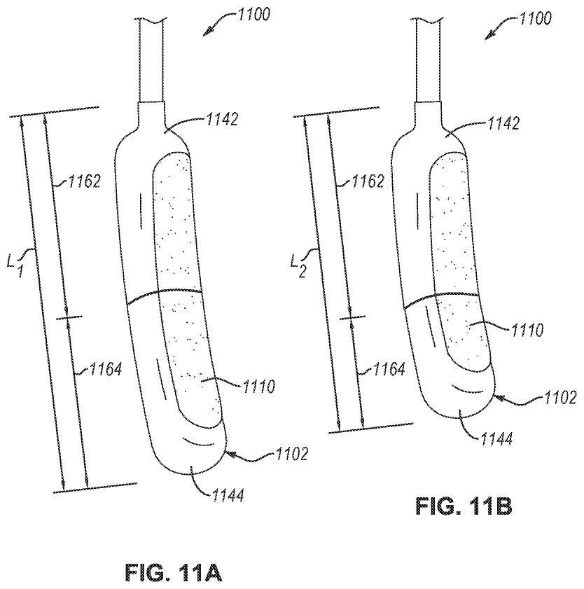

The size of the region about the urethral opening may vary from patient to patient. The variation in the size of the region about the urethral opening may mean that the size of the region about the urethral opening is too large or too small for certain fluid collection assemblies. The fluid collection assemblies that are too large or too small for the particular patient are likely to cause the bodily fluids to leak. In an example, a fluid collection assembly that is too large may abut anatomical features that cause the fluid collection assembly to be incorrectly positioned relative to the patient. In an example, a fluid collection assembly that is too small may be unable to sufficiently cover the region about the urethral opening to inhibit bodily fluid leaks. The fluid collection assemblies illustrated in FIGS. 11A-13 illustrate examples of fluid collection assemblies that are able to vary the lengths thereof such that the fluid collection assemblies may exhibit a size that corresponds to the size of the region about the urethral opening for each patient. For example, the fluid collection assemblies illustrated in FIGS. 11A-13 may be able to increase or decrease the lengths thereby by at most about 5%, at most about 10%, at most about 20%, at most about 30%, at most about 40%, at most about 50%, at most about 70%, at most about 90%, or in ranges of about 5% to about 20%, about 10% to about 30%, about 20% to about 40%, about 30% to about 50%, about 40% to about 70%, or about 50% to about 90%. The ability of the fluid collection assemblies to change a length thereof are leak prevention features since varying the length of the fluid collection assemblies may inhibit leaks of the bodily fluids caused by the fluid collection assembly being too larger or too small.

FIGS. 11A and 11B are isometric views of a fluid collection assembly 1100 exhibiting a first length L₁ and a second length L₂, respectively, according to an embodiment. Except as otherwise disclosed herein, the fluid collection assembly 1100 may be the same as or substantially similar to any of the fluid collection assemblies disclosed herein and may include any of the features of any of the fluid collection assemblies disclosed herein. For example, the fluid collection assembly 1100 may include a fluid impermeable barrier 1102 and at least one porous material 1110.

The fluid collection assembly 1100 includes a first region 1162 and a second region 1164. In an embodiment, as illustrated, the first region 1162 forms at least the proximal end region 1142 of the fluid collection assembly 1100 and the second region 1164 forms at least the distal end region 1144 of the fluid collection assembly 1100. Alternatively, the first region 1162 may form at least the distal end region 1144 of the fluid collection assembly 1100 and the second region 1164 may form at least the proximal end region 1142 of the fluid collection assembly 1100.

The first region 1162 and the second region 1164 are configured to move relative to each other (e.g., slidably engage each other). Moving the first region 1162 and the second region 1164 relative to each other allows the length of the fluid collection assembly 1100 to change. In an example, when the fluid collection assembly 1100 exhibits a first length L₁, the second region 1164 may be moved towards the first region 1162 to decrease the length of the fluid collection assembly 1100 from the first length L₁ to the second length L₂ (e.g., the second length L₂ is less than the first length L₁). In an example, when the fluid collection assembly 1100 exhibits the first length L₁, moving the second region 1164 away from the first region 1162 may increase the length of the fluid collection assembly 1100 to a third length (not shown) that is greater than the first length L₁ and second length L₂. In an example, when the fluid collection assembly exhibit the second length L₂, moving the second region 1164 away from the first region 1162 may increase the length of the fluid collection assembly 1100 to the first length L₁, an intermediate length between the first length L₁ and the second length L₂, or to the third length.

In an embodiment, a portion of one of the second region 1164 is configured to be positioned under a portion of the first region 1162 to facilitate movement of the first region 1162 and the second region 1164 relative to each other. In other words, a portion of the first and second regions 1162, 1164 are configured to overlap. The quantity of the second region 1164 that is positioned under the first region 1162 depends on the length of the fluid collection assembly 1100. The quantity of the second region 1164 that is positioned under the first region 1162 may change depending on whether the length of the fluid collection assembly 1100 is increasing or decreasing. For example, that quantity of the second region 1164 that is under the first region 1162 may decrease when the length of the fluid collection assembly 1100 increases and may increase when the length of the fluid collection assembly 1100 decreases. In a particular example, a first quantity of the second region 1162 may be positioned under the first region 1162 when the fluid collection assembly 1100 exhibits the first length L₁ and a second quantity of the second region 1162 may be positioned under the first region 1162 when the fluid collection assembly 1100 exhibits the second length L₂. The first quantity is less than the second quantity.

Disposing a portion of the second region 1164 under a portion of the first region 1162 may increase a width (e.g., radius) of the fluid collection assembly 1100 at and near the portion of the first and second regions 1162, 1162 that overlap. The overlap may also cause a compressive force to be applied to a portion of the porous material 1110 that is at least adjacent to the overlap which may decrease a volume of the chamber (e.g., decrease the quantity of bodily fluids that may be temporarily stored in the porous material 1110). Generally, increasing the overlap between the first and second regions 1162, 1164 decreases the volume of the chamber and, thus, decreases the quantity of bodily fluids that may be temporarily stored in the chamber. Accordingly, it may be beneficial to minimize the quantity of the second portion 1164 that is positioned under the first region 1162 to maximize the quantity of bodily fluids that may be temporarily stored in the fluid collection assembly 1100.

In an embodiment, as illustrated, the fluid impermeable barrier 1102 of the second region 1164 is configured to be positioned under the fluid impermeable barrier 1102 of the first region 1162 and the porous material 1110 of the second region 1164 is configured to be positioned under the porous material 1110 of the first region 1162. In such an embodiment, the volume of the fluid reservoir defined at the distal end region 1144 may remain unchanged. In an embodiment, the fluid impermeable barrier 1102 of the second region 1164 is configured to be positioned under the fluid impermeable barrier 1102 of the first region 1162 and the porous material 1110 of the second region 1164 is not configured to be positioned under the porous material 1110 of the first region 1162. In such an embodiment, decreasing a length of the fluid collection assembly 1100 may decrease a volume of the fluid reservoir.

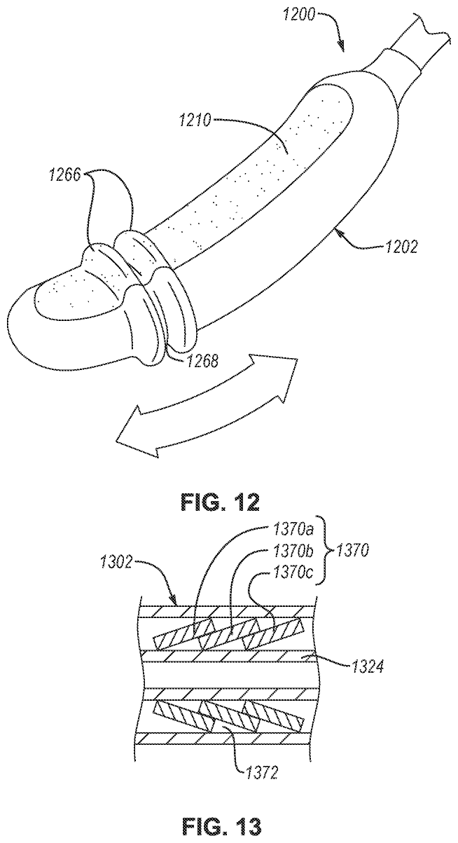

FIG. 12 is an isometric view of a fluid collection assembly 1200 that is configured to change a length thereof, according to an embodiment. Except as otherwise disclosed herein, the fluid collection assembly 1200 may be the same as or substantially similar to any of the fluid collection assemblies disclosed herein and may include any of the features of any of the fluid collection assemblies disclosed herein. For example, the fluid collection assembly 1200 may include a fluid impermeable barrier 1202 and at least one porous material 1210.

At least a portion of the fluid collection assembly 1200 includes a “crinkle structure” that is configured to allow the fluid collection assembly 1200 to change a length thereof. For example, at least one of the fluid impermeable barrier 1202 or the porous material 1210 may include the “crinkle structure.” The “crinkle structure” may include one or more peaks 1266 (e.g., a plurality of circumferentially extending peaks 1266 or one or more helically extending peaks 1266) and one or more valleys 1268 (e.g., a plurality of circumferentially extending valleys 1268 or one or more helically extending valleys 1268) disposed between portions of the peaks 1266. The distance between the peaks 1266 and valleys 1268 may be changes thereby allowing the length of the fluid collection assembly 1200 to change. For example, the distance between one or more of the peaks 1266 may be selectively increased or decreased by applying a compressive or tensile force to the fluid collection assembly 1200 that is generally parallel to a longitudinal axis of the fluid collection assembly 1200. The “crinkle structure” may be configured to maintain the distance between the peaks 1266 after the compressive or tensile force is removed from the fluid collection assembly 1200.

In an embodiment, the “crinkle structure” may also allow the fluid collection assembly 1200 to be selectively bent such that the fluid collection assembly 1200 exhibits a shape that generally corresponds to the shape of the region about the urethral opening. Such shaping of the fluid collection assembly 1200 may minimize the formation of gaps between the fluid collection assembly 1200 and the region about the urethral opening. The fluid collection assembly 1200 may be selectively bent by applying a force to the fluid collection assembly 1200 that causes the distance of the peaks 1266 on one side of the “crinkle structure” to increase or decrease while the distance between the same peaks 1266 on the opposing side of the “crinkle structure” remain substantially constant or changes at a rate that is different than the peaks 1266 on the other side.

In some embodiments, decreasing the volume of the chamber may also decrease the volume of the porous material. Decreasing the volume porous material may decrease the volume of the pores which may temporarily receive bodily fluids. FIG. 13 is a cross-sectional schematic of a portion of a fluid collection assembly 1300 that includes a porous material 1310 that is configured to minimize a decrease in the volume of the porous material when a length of the fluid collection assembly 1300 decreases, according to an embodiment. Except as otherwise disclosed herein, the fluid collection assembly 1300 may be the same as or substantially similar to any of the fluid collection assemblies disclosed herein and may include any of the features of any of the fluid collection assemblies disclosed herein. For example, the fluid collection assembly 1300 may include a fluid impermeable barrier 1302 and a conduit 1324.

The porous material includes a plurality of segments 1370. The plurality of segments 1370 are configured to move relative to each other (e.g., slidably engage each other) and to overlap with each other. For example, in the illustrated embodiment, the plurality of segments 1370 may include a first segment 1370a, a second segment 1370b, and a third segment 1370c. It is noted that the porous material may include more or less than the first, second, and third segments 1370a, 1370b, 1370c. A portion of the second segment 1370b is positioned under a portion the first segment 1370a, and the third segment 1370c is positioned under a portion of the second segment 1370b. The quantity of the second segment 1370b that is positioned under a portion the first segment 1370a, and the quantity of the third segment 1370c that is positioned under a portion of the second segment 1370b may vary depending on the length of the fluid collection assembly 1300. For example, increasing a length of the fluid collection assembly 1300 may decrease at least one of the quantity of the second segment 1370b that is positioned under a portion the first segment 1370a or the quantity of the third segment 1370c that is positioned under a portion of the second segment 1370b. Meanwhile, decreasing a length of the fluid collection assembly 1300 may increase at least one of the quantity of the second segment 1370b that is positioned under a portion the first segment 1370a or the quantity of the third segment 1370c that is positioned under a portion of the second segment 1370b.

The gap between the fluid impermeable barrier 1302 and the conduit 1324 may be selected to allow the plurality of segments 1370 to overlap without applying a significantly greater compressive force to the segments 1370 when the overlap between the segments 1370 increases thereby minimizing a decrease in a volume of the porous material. This gap may also allow the formation of unoccupied spaces 1372. The unoccupied spaces 1372 may increase the quantity of bodily fluids that may be temporarily stored in the chamber 1304.

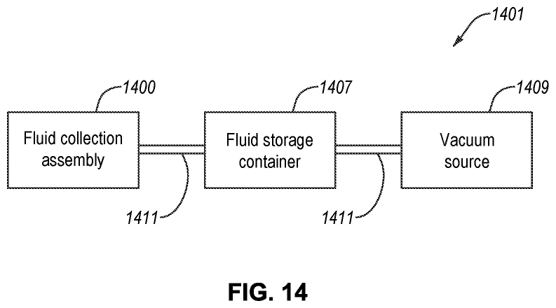

FIG. 14 is a block diagram of a system 1401 for fluid collection, according to an embodiment. The system 1401 includes a fluid collection assembly 1400, a fluid storage container 1407, and a vacuum source 1409. The fluid collection assembly 1400, the fluid storage container 1407, and the vacuum source 1409 may be fluidly coupled to each other via one or more conduits 1411. For example, fluid collection assembly 1400 may be operably coupled to one or more of the fluid storage container 1407 or the vacuum source 1409 via the conduit 1411. Fluid (e.g., urine or other bodily fluids) collected in the fluid collection assembly 1400 may be removed from the fluid collection assembly 1400 via the conduit 1411 which protrudes into the fluid collection assembly 1400. For example, an inlet of the conduit 1411 may extend into the fluid collection assembly 1400, such as to a fluid reservoir therein. The outlet of the conduit 1411 may extend into the fluid collection assembly 1400 or the vacuum source 1409. Suction force may be introduced into the chamber of the fluid collection assembly 1400 via the inlet of the conduit 1411 responsive to suction (e.g., vacuum) force applied at the outlet of the conduit 1411.

The suction force may be applied to the outlet of the conduit 1411 by the vacuum source 1409 either directly or indirectly. The suction force may be applied indirectly via the fluid storage container 1407. For example, the outlet of the conduit 1411 may be disposed within the fluid storage container 1407 and an additional conduit 1411 may extend from the fluid storage container 1407 to the vacuum source 1409. Accordingly, the vacuum source 1409 may apply suction to the fluid collection assembly 1400 via the fluid storage container 1407. The suction force may be applied directly via the vacuum source 1409. For example, the outlet of the conduit 1411 may be disposed within the vacuum source 1409. An additional conduit 1411 may extend from the vacuum source 1409 to a point outside of the fluid collection assembly 1400, such as to the fluid storage container 1407. In such examples, the vacuum source 1409 may be disposed between the fluid collection assembly 1400 and the fluid storage container 1407.

The fluid collection assembly 1400 may be similar or identical to any of the fluid collection assemblies disclosed herein in one or more aspects. The fluid collection assembly 1400 may be shaped and sized to be positioned adjacent to a female urethral opening. For example, the fluid collection assembly 1400 may include a fluid impermeable barrier at least partially defining a chamber (e.g., interior region) of the fluid collection assembly 1400. The fluid impermeable barrier also defines at least one opening extending therethrough from the external environment. The opening may be positioned adjacent to the female urethral opening. The fluid collection assembly 1400 may include porous material disposed in the chamber, such as one or more of a fluid permeable support and a fluid permeable membrane. The fluid collection assembly 1400 includes at least one leak prevention feature, as discussed herein. The conduit 1411 may extend into the fluid collection assembly 1400 at a first end (e.g., proximal) region, through one or more of the fluid impermeable barrier or the porous material to a second end region of the fluid collection assembly 1400. The conduit 1411 includes an inlet and an outlet, the outlet being fluidly coupled to the fluid storage container 1407 and the inlet being positioned in a portion of the chamber selected to be at a gravimetrically low point of the fluid collection assembly 1400 when worn.

The fluid storage container 1407 is sized and shaped to retain a fluid therein. The fluid storage container 1407 may include a bag (e.g., drainage bag), a bottle or cup (e.g., collection jar), or any other enclosed container for storing bodily fluid(s) such as urine. In some examples, the conduit 1411 may extend from the fluid collection assembly 1400 and attach to the fluid storage container 1407 at a first point therein. An additional conduit 1411 may attach to the fluid storage container 1407 at a second point thereon and may extend and attach to the vacuum source 1409. Accordingly, a vacuum (e.g., suction) may be drawn through fluid collection assembly 1400 via the fluid storage container 1407. Fluid, such as urine, may be drained from the fluid collection assembly 1400 using the vacuum source 1409.

The vacuum source 1409 may include one or more of a manual vacuum pump, and electric vacuum pump, a diaphragm pump, a centrifugal pump, a displacement pump, a magnetically driven pump, a peristaltic pump, or any pump configured to produce a vacuum. The vacuum source 1409 may provide a vacuum or suction to remove fluid from the fluid collection assembly 1400. In some examples, the vacuum source 1409 may be powered by one or more of a power cord (e.g., connected to a power socket), one or more batteries, or even manual power (e.g., a hand operated vacuum pump). In some examples, the vacuum source 1409 may be sized and shaped to fit outside of, on, or within the fluid collection assembly 1400. For example, the vacuum source 1409 may include one or more miniaturized pumps or one or more micro pumps. The vacuum sources 1409 disclosed herein may include one or more of a switch, a button, a plug, a remote, or any other device suitable to activate the vacuum source 1409.

The fluid collection assemblies disclosed herein are configured to collect one or more bodily fluids from a female urethral opening. However, it is noted that any of the concepts disclosed herein, such as any of the leak prevention features disclosed herein, may be used in fluid collection assemblies configured to collect bodily fluids from a male urethral opening (e.g., penis). Examples of fluid collection assemblies that are configured to collected bodily fluids from a male urethral opening and methods of using such fluid collection assemblies are disclosed in International Application No. PCT/US20/42262 filed on Jul. 14, 2020, U.S. patent application Ser. No. 16/433,773 filed on Apr. 3, 2020, and U.S. Provisional Patent Application No. 63/047,374 filed on Jul. 2, 2020, the disclosure of each of which is incorporated herein, in its entirety, by this reference.

While various aspects and embodiments have been disclosed herein, other aspects and embodiments are contemplated. The various aspects and embodiments disclosed herein are for purposes of illustration and are not intended to be limiting.

Terms of degree (e.g., “about,” “substantially,” “generally,” etc.) indicate structurally or functionally insignificant variations. In an example, when the term of degree is included with a term indicating quantity, the term of degree is interpreted to mean±10%, ±5%, or +2% of the term indicating quantity. In an example, when the term of degree is used to modify a shape, the term of degree indicates that the shape being modified by the term of degree has the appearance of the disclosed shape. For instance, the term of degree may be used to indicate that the shape may have rounded corners instead of sharp corners, curved edges instead of straight edges, one or more protrusions extending therefrom, is oblong, is the same as the disclosed shape, etc.

1. A fluid collection assembly, comprising:
a fluid impermeable barrier at least defining a chamber, at least one opening configured to be positioned adjacent to a urethral opening, and a fluid outlet;
at least one porous material disposed in the chamber and extending across the at least one opening; and
one or more leak prevention features including at least one of:
two wings extending from opposing portions of the fluid impermeable barrier, the two wings configured to contact thighs of an individual, the two wings configured as springs that flex and bend;
at least one absorbent element, at least a portion of the at least one absorbent element disposed on at least a portion of at least one exterior surface of the fluid impermeable barrier, the at least one absorbent element including at least one of fabric, gauze, cellulose, polypropylene, sponges, or super absorbent polymer; or
the fluid impermeable barrier including at least one ridge extending into the chamber from a portion of at least one interior surface of the fluid impermeable barrier.

2. The fluid collection assembly of claim 1, wherein the one or more leak prevention features include the fluid collection assembly exhibiting a generally triangular cross-sectional shape, and wherein a portion of the at least one porous material extends outwardly from the at least one opening and forms at least an apex of the generally triangular cross-sectional shape.

3. The fluid collection assembly of claim 1, wherein the one or more leak prevention features includes the fluid collection assembly exhibiting a generally triangular cross-sectional shape, a portion of the at least one porous material extending across the at least one opening forming at least an edge of the generally triangular cross-sectional shape.

4. The fluid collection assembly of claim 1, wherein the one or more leak prevention features includes the two wings, the two wings extending from or near opposing the portions of the fluid impermeable barrier that define the at least one opening.

5. The fluid collection assembly of claim 4, wherein the two wings include:
an outer layer defining a cavity; and
at least one super absorbent polymer disposed in the cavity.

6. The fluid collection assembly of claim 4, wherein the two wings include one or more reinforcement features that exhibit a Young's modulus that is greater than the rest of the two wings.

7. The fluid collection assembly of claim 4, wherein the two wings exhibit at least one concavely curved portion, as viewed from the at least one opening.

8. The fluid collection assembly of claim 1, wherein the one or more leak prevention features includes a portion of the porous material extending across the at least one opening that exhibits a concave shape.

9. The fluid collection assembly of claim 1, wherein:
the fluid collection assembly includes a proximal end region and a distal end region opposite the proximal end region; and
the one or more leak prevention features includes a width of the fluid impermeable barrier measured perpendicular to a longitudinal axis of the chamber decreasing from the proximal end region to the distal end region.

10. The fluid collection assembly of claim 1, wherein the one or more leak prevention features includes the at least one adsorbent element.

11. The fluid collection assembly of claim 10, wherein the at least one absorbent element is disposed on a portion of the at least one exterior surface that defines the opening.

12. The fluid collection assembly of claim 10, wherein a portion of the at least one adsorbent element is disposed in the chamber.

13. The fluid collection assembly of claim 10, wherein at least a portion of the at least one absorbent element is disposed on an external surface of at least a portion of a distal end region of the fluid collection assembly.

14. The fluid collection assembly of claim 1, wherein the one or more leak prevention features includes a base configured to have an individual disposed thereon, the fluid impermeable barrier secured to the platform.

15. The fluid collection assembly of claim 1, wherein the one or more leak prevention features include a fluid impermeable barrier configured to change a length thereof by about 5% to about 50%.

16. The fluid collection assembly of claim 15, wherein the fluid impermeable barrier includes a first region and a second region, a portion of the second region positioned under the first region, the first region and second region configured to slidably engage each other.

17. The fluid collection assembly of claim 15, wherein at least a portion of the fluid impermeable barrier includes one or more peaks and one or more valleys.

18. The fluid collection assembly of claim 15, wherein the at least one porous material includes a plurality of segments configured to slidably engage each other.

19. The fluid collection assembly of claim 1, wherein the fluid impermeable barrier includes the at least one ridge.

20. A system comprising:
a fluid collection assembly; comprising:
a fluid impermeable barrier at least defining a chamber, at least one opening configured to be positioned adjacent to a urethral opening, and a fluid outlet;
at least one porous material disposed in the chamber and extending across the at least one opening; and
one or more leak prevention features including at least one of:
two wings extending from opposing portions of the fluid impermeable barrier, the two wings configured as springs that flex and bend;
at least one absorbent element, at least a portion of the at least one absorbent element disposed on at least a portion of at least one exterior surface of the fluid impermeable barrier, the at least one absorbent element including at least one of fabric, gauze, cellulose, polypropylene, sponges, or super absorbent polymer; or
the fluid impermeable barrier including at least one ridge extending into the chamber from a portion of at least one interior surface of the fluid impermeable barrier;
a fluid storage container; and
a vacuum source configured to supply a suction force to the fluid collection assembly;
wherein the chamber, the fluid storage container, and the vacuum source are in fluid communication with each other via at least one conduit.
